(19) United States
(12) Patent Application Publication
(10) Pub. No.: US 2026/0217685 A1
(43) Pub. Date: Jul. 30, 2026

(54) DEUTERATED COMPOUNDS

(71) Applicant: Sun Pharmaceutical Industries, Inc., Princeton, NJ (US)

(72) Inventors: Brett D. GROTBECK, Lexington, MA (US); Jenisha GANDHI, Lexington, MA (US); Nancy DEGRACE, Lunenburg, MA (US); Patrick BAZINET, Somerville, MA (US)

(21) Appl. No.: 19/273,809

(22) Filed: Jul. 18, 2025

Related U.S. Application Data
(60) Provisional application No. 63/749,116, filed on Jan. 24, 2025.

(51) Int. Cl. C07D 403/04; A61K 31/437; A61K 31/4985; A61K 31/506; A61K 31/519; A61K 31/529; A61K 31/5377; A61K 31/541; C07B 59/00

(52) U.S. Cl. CPC C07D 403/04; A61K 31/437; A61K 31/4985; A61K 31/506; A61K 31/519; A61K 31/529; A61K 31/5377; A61K 31/541; C07B 59/002; C07B 2200/05

(57) ABSTRACT

The present disclosure is directed to deuterated compounds of the general formula:

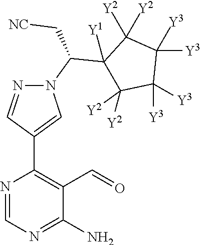

and pharmaceutically acceptable salts thereof. Also disclosed herein are pharmaceutical compositions comprising the deuterated compound.

### FIELD OF THE INVENTION

The present disclosure is directed to deuterated compounds of the general formula:

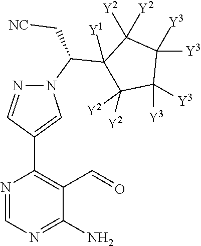

and pharmaceutically acceptable salts thereof. Also disclosed herein are pharmaceutical compositions comprising the deuterated compound.

### BACKGROUND OF THE INVENTION

Janus Associated Kinases (JAKs) JAK1, JAK2, JAK3, and TYK2 are known to mediate the signaling of a number of cytokines and growth factors important for hematopoiesis and immune function, and JAK signaling has been associated with various diseases, such as cancers and immune disorders. JAK signaling involves recruitment of STATs (signal transducers and activators of transcription) to cytokine receptors, activation and subsequent localization of STATs to the nucleus leading to modulation of gene expression.

Several JAK inhibitors have been approved and are currently in clinical trials. Examples of JAK inhibitors include the approved drugs deuruxolitinib, ruxolitinib, filgotinib, baricitinib, momelotinib, pacritinib, and tofacitinib among others. JAK inhibitors can differ according to the selectivity against different JAK subtypes. For instance, ruxolitinib and baricitinib are selective dual inhibitors of JAKI and JAK2, while tofacitinib is selective for the JAK3 subtype. JAK inhibitors have reported utility for various cancer and autoimmune diseases. Ruxolitinib is currently approved for the treatment of patients with intermediate or high-risk myelofibrosis, including primary myelofibrosis, post-polycythemia vera myelofibrosis, post-essential thrombocythemia myelofibrosis, and steroid-refractory acute graft-versus-host disease, and is in clinical trials for various other conditions. Tofacitinib and baricitinib are approved for rheumatoid arthritis. Deuruxolitinib phosphate (“CTP-543”), known by the chemical name 1H-pyrazole-1-propanenitrile, β-(cyclopentyl-2,2,3,3,4,4,5,5-d₈)-4-(7H-pyrrolo[2,3-d]pyrimidin-4-yl)-, (βR)-, phosphate (1:1), is approved for the treatment of alopecia areata.

### SUMMARY OF THE INVENTION

The present disclosure provides a compound represented by Formula (I)

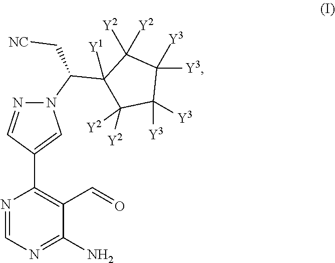

or a pharmaceutically acceptable salt thereof, wherein
Y¹ is selected from hydrogen and deuterium;
each Y² is independently selected from hydrogen and deuterium; and
each Y³ is independently selected from hydrogen and deuterium;
provided that at least one of Y¹, Y², and Y³ is deuterium; and
wherein each position designated specifically as deuterium has at least 95% deuterium incorporation.

In some embodiments, each Y² is the same. In some embodiments, each Y³ is the same.

In some embodiments, the compound is selected from any of Formulas (II)-(VI):

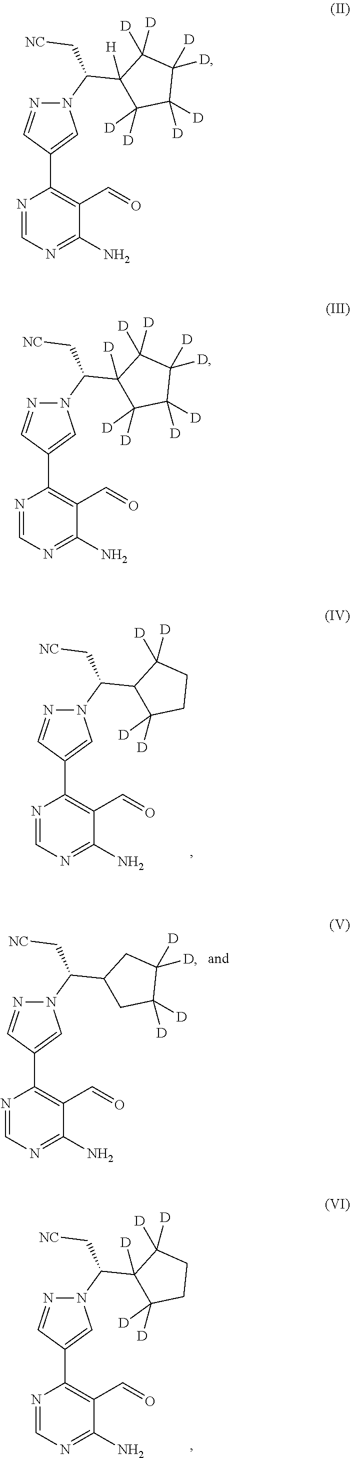

or a pharmaceutically acceptable salt thereof.

In some embodiments, the compound is Formula (II):

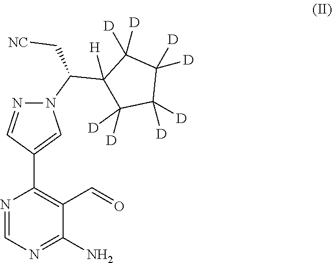 [SMILES: [2H]C1([2H])C([C@@H](CC#N)n2cc(-c3ncnc(N)c3C=O)cn2)C([2H])([2H])C([2H])([2H])C1([2H])[2H]]

or a pharmaceutically acceptable salt thereof.

In some embodiments, each position designated specifically as deuterium in Formula (I) has at least 97% deuterium incorporation.

In some embodiments, the pharmaceutically acceptable salt is a phosphate salt.

In some embodiments, the disclosure provides a pharmaceutical composition comprising a compound represented by the following structural formula:

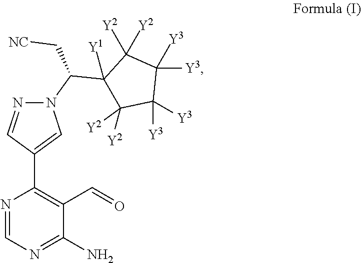

or a pharmaceutically acceptable salt thereof,
   wherein Y¹ is selected from hydrogen and deuterium;
   each Y² is independently selected from hydrogen and deuterium;
   each Y³ is independently selected from hydrogen and deuterium;
   provided that at least one of Y² and Y³ is deuterium; and
      wherein each position designated specifically as deuterium has at least 95% deuterium incorporation; and a pharmaceutically acceptable carrier.

In some embodiments, each Y² is the same. In some embodiments, each Y³ is the same.

In some embodiments, the pharmaceutical composition further comprises an additional therapeutic agent which is a JAK inhibitor. In some embodiments, the additional therapeutic agent which is a JAK inhibitor is selected from CTP-543, ruxolitinib, tofacitinib, baricitinib, upadacitinib, fedratinib, filgotinib, momelotinib, pacritinib, itacitinib, peficitinib, PF-06651600, PF-06700841 (brepocitinib), and abrocitinib, or a pharmaceutically acceptable salt thereof.

In some embodiments, the composition comprises a compound of Formula (II)

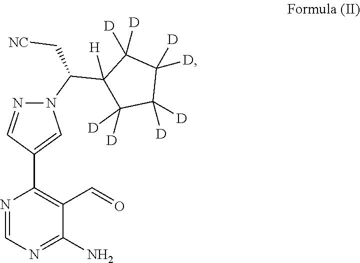

or a pharmaceutically acceptable salt thereof, and CTP-543, or a pharmaceutically acceptable salt thereof.

In some embodiments, the pharmaceutical composition is formulated in capsule form. In some embodiments, the pharmaceutical composition is formulated in tablet form.

In some embodiments, each position designated specifically as deuterium in Formula (I) has at least 97% deuterium incorporation.

In some embodiments, the compound of Formula (I) is present in an amount less than 1% w/w of the pharmaceutical composition. In some embodiments, the compound of Formula (I) is present in an amount less than 0.5% w/w of the pharmaceutical composition.

In some embodiments, the pharmaceutical composition further comprises povidone In some embodiments, the povidone is povidone K30 LP.

In some embodiments, the compound of Formula (I) is present in an amount less than 0.05% w/w of the pharmaceutical composition.

In some embodiments, the disclosure provides method of inhibiting the activity of one or more of JAK1 or JAK2 in a cell, comprising contacting the cell with a composition as described herein, or a pharmaceutically acceptable salt thereof.

In some embodiments, the method further comprises contacting the cell with a composition comprising one or more JAK inhibitors selected from CTP-543, ruxolitinib, tofacitinib, baricitinib, upadacitinib, fedratinib, filgotinib, momelotinib, pacritinib, itacitinib, peficitinib, PF-06651600, PF-06700841 (brepocitinib), and abrocitinib; or a pharmaceutically acceptable salt thereof.

In some embodiments, the disclosure provides a method of treating a disease or disorder in a human subject that is beneficially treated by inhibiting the activity of a JAK, the method comprising administering to the subject an effective amount of a pharmaceutical composition as described herein.

In some embodiments, the disease or disorder is a hair loss disorder selected from alopecia areata, alopecia totalis, alopecia universalis, ophiasis, androgenic alopecia, and telogen effluvium. In some embodiments, the hair loss disorder is alopecia areata.

In some embodiments, the pharmaceutically acceptable salt is a phosphate salt.

In some embodiments, the disclosure provides a method of treating a disease or disorder that is beneficially treated by inhibiting the activity of a JAK in a human subject in need thereof, comprising administering to the human subject an effective amount of a pharmaceutical composition as described herein.

In some embodiments, the JAK inhibitor is selected from CTP-543, ruxolitinib, tofacitinib, baricitinib, upadacitinib, fedratinib, filgotinib, momelotinib, pacritinib, itacitinib, peficitinib, PF-06651600, PF-06700841 (brepocitinib), and abrocitinib, and pharmaceutically acceptable salts thereof.

In some embodiments, the pharmaceutical composition comprises povidone K30 LP.

In some embodiments, the compound is present in the pharmaceutical composition at less than 0.05% w/w. In some embodiments, the compound of Formula (I) is a compound of Formula (II)

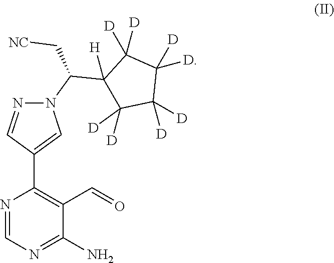

or a pharmaceutically acceptable salt thereof, and the second JAK inhibitor is CTP-543, or a pharmaceutically acceptable salt thereof.

In some embodiments, the pharmaceutical composition is administered at 16 mg/day or 24 mg/day of CTP-543.

In some embodiments, each position designated specifically as deuterium has at least 97% deuterium incorporation.

In some embodiments, the pharmaceutically acceptable salt is a phosphate salt.

### BRIEF DESCRIPTION OF THE DRAWINGS

FIG. 1 depicts the chromatogram from a reverse-phase High Pressure Liquid Chromatography (HPLC) run on the oxidation reaction product described in the first instance of Example 1. The peaks for both CTP-543 and the Formula (II) compound are indicated. The y-axis represents the absorbance units, whereas the x-axis represents the retention time in minutes.

FIG. 2 depicts the chromatogram from an HPLC run on the oxidation reaction product described in the second instance of Example 1. The peaks for both CTP-543 and the Formula (II) compound are indicated. The y-axis represents the absorbance units, whereas the x-axis represents the retention time in minutes.

### DETAILED DESCRIPTION OF THE INVENTION

The present disclosure relates to deuterated compounds similar to deuruxolitnib and their pharmaceutically acceptable salts. In some embodiments, the deuterated compounds of the disclosure are represented by the following structure Formula (I):

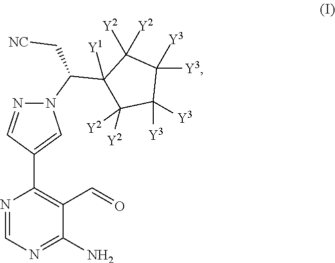

or a pharmaceutically acceptable salt thereof, wherein Y¹ is selected from hydrogen and deuterium; each Y² is independently selected from hydrogen and deuterium; and each Y³ is independently selected from hydrogen and deuterium. In some embodiments, at least one of Y¹, Y², and Y³ is deuterium. In some embodiments, each Y² is the same. In some embodiments, each Y² at one carbon position on the cyclopentyl ring is two hydrogens and each Y² at the other carbon position on the cyclopentyl ring is two deuterium. In some embodiments, at one or both carbon positions on the cyclopentyl ring, one Y² is a hydrogen and one Y² is a deuterium. In some embodiments, each Y³ is the same. In some embodiments, each Y³ at one carbon position on the cyclopentyl ring is two hydrogens and each Y³ at the other carbon position on the cyclopentyl ring is two deuterium. In some embodiments, at one or both carbon positions on the cyclopentyl ring, one Y³ is a hydrogen and one Y³ is a deuterium.

One of skill in the art will understand that for each of the carbon positions shown in Formula (I), or any of the Formulas (II)-(VII) of the disclosure, as deuterium (i.e., “D”), deuterium may not be incorporated in 100% of the positions and would still fall within the scope of Formula (I). In some embodiments, at least 90% of each specified deuterated position of Formula (I) comprises deuterium, or at least 91%, at least 92%, at least 93%, at least 94%, at least 95%, at least 96%, at least 97%, at least 98%, or at least 99% of each specified deuterated position of Formula (I) comprises deuterium as determined by 1H-NMR analysis.

Also provided herein are pharmaceutical compositions that contain a compound of Formula (I). In some embodiments, the pharmaceutical composition contains a compound of Formula (I) and an additional therapeutic agent which is a JAK inhibitor. In some embodiments, the additional therapeutic agent is a JAK inhibitor selected from CTP-543, ruxolitinib, tofacitinib, baricitinib, upadacitinib, fedratinib, filgotinib, momelotinib, pacritinib, itacitinib, peficitinib, PF-06651600, PF-06700841 (brepocitinib), and abrocitinib, or a pharmaceutically acceptable salt thereof. In some embodiments, the additional therapeutic agent in the pharmaceutical composition contributes to at least 95% of the inhibition of JAK signaling (e.g., inhibition of JAK1, JAK2, JAK3, and/or Tyk2), or at least 99% of the inhibition of JAK signaling, or at least 99.9% of the inhibition of JAK signaling.

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. As used herein, “a” or “an” may mean one or more. As used herein, when used in conjunction with the word “comprising,” the words “a” or “an” may mean one or more than one. As used herein, “another” or “a further” may mean at least a second or more.

Throughout this application, the term “about” is used to indicate that a value includes the inherent variation of error for the compositions or methods being employed to determine the value, or the variation that exists. Typically, the term “about” is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability, depending on the situation.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

The terms “administration” or “administering” as used herein refer to providing, contacting, and/or delivering a compound or compounds by any appropriate route to achieve the desired effect. Administration may include, but is not limited to, oral, sublingual, parenteral topical, buccal, rectal, or vaginal. In some embodiments, the compounds and compositions described herein are suitable for oral administration.

As used herein, the terms “carrier” or “carrier materials” refer to one or more non-active, non-toxic pharmaceutically acceptable carriers, binders, diluents, excipients, or adjuvants. The carrier materials are acceptable in the sense of being compatible with the other ingredients of the composition and are not deleterious to the recipient. The pharmaceutical compositions of the present disclosure can be adapted for administration by any suitable route by selection of appropriate carrier materials and a dosage of a compound of the disclosure effective for the treatment intended. For example, the compositions are prepared in a form suitable for oral administration. Accordingly, the carrier material employed can be a solid, and may be formulated with the compound as a unit-dose composition, for example, a tablet or a capsule. Such pharmaceutical compositions of the disclosure can be prepared by any of the well-known techniques of pharmacy, consisting essentially of admixing the components.

The terms “drug,” “active agent,” “therapeutic agent,” “API,”, or “pharmaceutical,” may be used interchangeably herein to refer to a compound, agent, or substance that has measurable specified or selected physiological activity when administered to a subject in an effective amount. In some embodiments, for convenience the terms “drug,” “active agent,” “therapeutic agent,” “API,”, or “pharmaceutical,” can refer to a pharmaceutically acceptable salt of such compound, agent, or substance, even if the phrase pharmaceutically acceptable salt thereof is not specifically recited. In some embodiments, the terms “drug,” “active agent,” “therapeutic agent,” “API,”, or “pharmaceutical” can include the compound of any one of Formulas (I)-(V), or pharmaceutically acceptable salt thereof, even if no specified or selected physiological activity is measured.

The term “oxidation reaction” or “oxidizing” means a chemical reaction in which a substance or functional group reacts with an agent (i.e., an oxidizing agent) resulting in the loss of electrons and hydrogen atoms or in the addition of oxygen atoms or other electronegative heteroatoms or both.

The term “pharmaceutically acceptable” as used herein, refers to a component that is, within the scope of sound medical judgement, suitable for use in contact with the tissues of humans and other mammals without undue toxicity, irritation, allergic response, and the like, and are commensurate with a reasonable benefit/risk ratio. A “pharmaceutically acceptable salt” means any non-toxic salt that, upon administration to the recipient, is capable of providing, either directly or indirectly, a compound of this disclosure.

The term “pharmaceutical composition” as used herein refer to a compound or composition capable of inducing a desired therapeutic effect when properly administered to a subject. In some embodiments, the term “pharmaceutical composition” can include the compound of any one of Formulas (I)-(V).

As used herein, the term “room temperature” generally refers to 4° C. to 30° C., 18° C. to 22° C., 19° C. to 22° C., 20° C. to 22° C. or 20±5° C.

The term “treating” or “treatment” refers to administering a compound or pharmaceutical composition to a subject in order to affect an alteration or improvement of a disease, disorder, or condition in the subject. The terms “treatment,” “treat,” and “treating” as used herein may refer to both therapeutic treatment and prophylactic or preventative measures or to partially or completely alleviating, inhibiting, delaying onset of, preventing, ameliorating, and/or relieving a disease or disorder, or one or more symptoms of the disease or disorder, as described herein. Those in need of treatment include subjects having a disease or condition, such as, but not limited to a JAK-associated disease or condition, as well as those prone to having disease or condition or those for which a disease or condition is to be prevented.

The term “therapeutically effective amount” as used herein refers to the amount of active compound or pharmaceutical agent that elicits a biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease, (2) inhibiting the disease; for example, inhibiting a disease, condition or disorder in an individual that is experiencing or displaying the pathology or symptomatology of the disease, condition or disorder (i.e., arresting further development of the pathology and/or symptomatology), and (3) ameliorating the disease; for example, ameliorating a disease, condition or disorder in an individual that is experiencing or displaying the pathology or symptomatology of the disease, condition or disorder (i.e., reversing the pathology and/or symptomatology).

The term “JAK inhibitors” refers to therapeutic agents or pharmaceutically acceptable salts thereof useful in treatment of JAK1, JAK2, JAK3 and/or TYK2-mediated diseases and conditions, including, but not limited to CTP-543, ruxolitinib, tofacitinib, baricitinib, upadacitinib, fedratinib, filgotinib, momelotinib, pacritinib, itacitinib, peficitinib, PF-06651600, PF-06700841 (brepocitinib), and abrocitinib. The term “JAK signaling” refers to the JAK-STAT pathway mediated, in part, by Janus kinases (JAKs).

The term “stereoisomer” as used herein can refer to both enantiomers and diastereomers, or to a specific enantiomer and/or diastereomer. In some embodiments, the compound of any of Formulas (I)-(VI) is the (R)-enantiomer, substantially free of the(S)-enantiomer. In some embodiments, the compound of any of Formulas (I)-(VI) is the(S)-enantiomer, substantially free of the (R)-enantiomer.

In some embodiments, the term “subject” is used herein to refer to a mammalian subject. In some embodiments, the “subject” is a human, domestic animals, farm animals, sports animals, and zoo animals, e.g., humans, non-human primates, dogs, cats, guinea pigs, rabbits, rats, mice, horses, cattle, etc. In some embodiments, the subject is a human. In some embodiments, the human subject is an adult (i.e., at least 18 years old). In some embodiments, the human subject is an adolescent (i.e., from 12 years to less than 18 years old). In some embodiments, the human subject is a pediatric subject (i.e., from 6 years to less than 12 years old). In some embodiments, the human subject is a male. In some embodiments, the human subject is a female.

### Compounds

In some embodiments, the disclosure provides a compound of the following structural Formula (I):

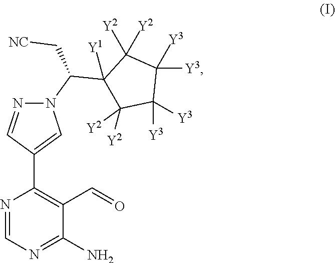

or a pharmaceutically acceptable salt thereof, wherein Y¹ is selected from hydrogen and deuterium; each Y² is independently selected from hydrogen and deuterium; and each Y³ is independently selected from hydrogen and deuterium. In some embodiments, each Y² is the same. In some embodiments, each Y² at one carbon position on the cyclopentyl ring is two hydrogens and each Y² at the other carbon position on the cyclopentyl ring is two deuterium. In some embodiments, at one or both carbon positions on the cyclopentyl ring, one Y² is a hydrogen and one Y² is a deuterium. In some embodiments, each Y³ is the same. In some embodiments, each Y³ at one carbon position on the cyclopentyl ring is two hydrogens and each Y³ at the other carbon position on the cyclopentyl ring is two deuterium. In some embodiments, at one or both carbon positions on the cyclopentyl ring, one Y³ is a hydrogen and one Y³ is a deuterium. In some embodiments, at least one of Y¹, Y², and Y³ is deuterium. In some embodiments, Y¹ is hydrogen. In some embodiments, each Y² and each Y³ is deuterium. In some embodiments, Y¹ is deuterium, and each Y² and each Y³ is deuterium. In some embodiments, Y¹ is hydrogen, each Y² is hydrogen, and each Y³ is deuterium. In some embodiments, Y¹ is hydrogen, each Y² is deuterium, and each Y³ is hydrogen.

In some embodiments, when Y¹ is hydrogen, the position incorporates less than 2% deuterium, or less than 1% deuterium, or less than 0.5% deuterium. In some embodiments, Y¹ is deuterium. In some embodiments, one or both Y² is hydrogen. In some embodiments, when Y² is hydrogen, the position incorporates less than 2% deuterium, or less than 1% deuterium, or less than 0.5% deuterium. In some embodiments, each Y² is deuterium. In some embodiments, one or both Y³ is hydrogen. In some embodiments, when Y³ is hydrogen, the position incorporates less than 2% deuterium, or less than 1% deuterium, or less than 0.5% deuterium. In some embodiments, each Y³ is deuterium. In some embodiments, each Y² and each Y³ is deuterium. In some embodiments, Y¹ is deuterium, and each Y² and each Y³ is deuterium. In some embodiments, Y¹ is hydrogen, and each Y² and each Y³ is deuterium. In some embodiments, Y¹ is hydrogen, each Y² is hydrogen, and each Y³ is deuterium. In some embodiments, Y¹ is hydrogen, each Y² is deuterium, and each Y³ is deuterium.

In some embodiments, the compound is selected from any of Formulas (II)-(VI):

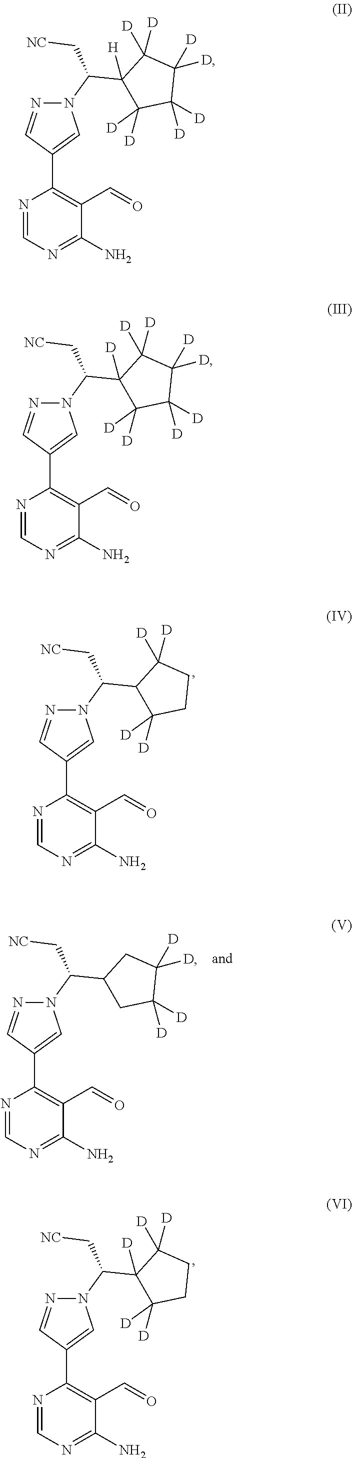

or a pharmaceutically acceptable salt thereof. In some embodiments, the pharmaceutically acceptable salt of the compound of Formulas (II)-(VI) is a phosphate salt.

In some embodiments, the compound is of Formula (II):

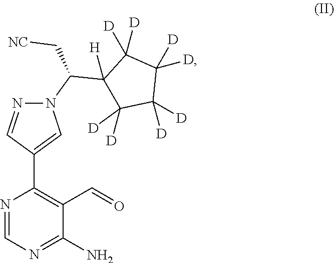

or a pharmaceutically acceptable salt thereof. The compound of Formula (II) is also known as (R)-3-(4-(6-amino-5-formylpyrimidin-4-yl)-1H-pyrazol-1-yl)-3 (cyclopentyl-2,2,3,3,4,4,5,5,-d₈) propanenitrile. In some embodiments, the pharmaceutically acceptable salt of the compound of Formulas (II) is a phosphate salt.

In some embodiments, in any of the compounds of Formulas (I)-(VI), each position designated specifically as deuterium has at least 90% deuterium incorporation, e.g., 90%, 91%, 92%, 93%, 94%, 95%, 96%, 97%, 98%, 99%, 99.5%, or more deuterium incorporation. In some embodiments, each position designated specifically as deuterium has at least 95% deuterium incorporation. In some embodiments, each position designated specifically as deuterium has at least 97% deuterium incorporation. In some embodiments, each position designated specifically as deuterium has at least 99% deuterium incorporation.

In some embodiments, a compound of Formula (I), (II), (III), (IV), (V), or (VI), or pharmaceutically acceptable salt, is an isolated compound of at least 90% purity, or at least 95% purity, at least 97% purity, at least 98% purity, or at least 99% purity. In some embodiments, purity can be measured by HPLC (e.g., Waters XBridge C18, 4.6×150 mm, 3.5 μm column (Waters, PIN 186003034); mobile phases composed of 20 mM ammonium acetate, pH 8.55 in water, acetonitrile, and isopropanol; under gradient elution conditions; flow rate of 1.0 mL/min; column temperature of 30° C.; detection by ultraviolet (UV) absorbance at 254 nm; retention time of approximately 14-16 minutes for compounds of the present disclosure, e.g., Formula (II)

In some embodiments, the compound of any of Formulas (I)-(VI) is the (R)-enantiomer, substantially free of the(S)-enantiomer. Enantiomeric purity can be assessed by art-recognized methods such as chiral HPLC. In certain embodiments, the compound of Formula (I) is the (R)-enantiomer with an enantiomeric excess (e.e.) of at least 95% e.e., or at least 98% e.e., or at least 99% e.e.

In some embodiments, each position of a compound of Formula (I) designated specifically as deuterium has at least 90% deuterium incorporation, at least 95% deuterium incorporation, at least 97% deuterium incorporation, at least 98% deuterium incorporation, or at least 99% deuterium incorporation. In some embodiments, the pharmaceutically acceptable salt is a phosphate salt.

In some embodiments, any atom not designated as deuterium is present at its natural isotopic abundance in compounds of Formula (I), Formula (II), Formula (III), Formula (IV), Formula (V), Formula (VI), or a pharmaceutically acceptable salt thereof.

In some embodiments, the compound is the pharmaceutically acceptable salt form of a compound of any of Formulas (I)-(VI). Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Thus, suitable pharmaceutically acceptable salts of the disclosure include, but are not limited to, sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate and mandelate salts. In some embodiments, the pharmaceutically acceptable salt is sulfate, phosphate, maleate, or any combination thereof. In some embodiments, the pharmaceutically acceptable salt form of the compound is the phosphate salt form.

Compounds of Formula (I)-(VI) can be prepared by a variety of methods. For example, a compound of Formula (II) can be prepared from e.g., CTP-543 as described in Example 1. In some embodiments, a compound of any one of Formulas (I)-(VI) can be obtained by eluting from a composition comprising a JAK inhibitor, such as CTP-543, after exposure to humidity, oxygen, and a particular temperature for a period of time. In some embodiments, the humidity is about 0% humidity to about 100% humidity, about 10% humidity to about 90% humidity, about 20% humidity to about 90% humidity, about 30% humidity to about 90% humidity, about 40% humidity to about 100% humidity, about 50% humidity to about 90% humidity, about 60% humidity to about 90% humidity, 65% to about 85% relatively humidity, or about 65% to about 80% relatively humidity, or about 65% to about 75% relative humidity, or about 65% to about 70% relatively humidity, or about 70% to about 85% relatively humidity, or about 75% to about 85% relatively humidity, or about 65% relatively humidity, or about 70% relatively humidity, or about 75% relatively humidity. In some embodiments, the temperature is room temperature (e.g., about 20° C. to about 30° C.). In some embodiments the temperature is about 30° C. to about 50° C., or about 30° C. to about 45° C., or about 30° C. to about 40° C., or about 35° C. to about 50° C., or about 40° C. to about 50° C., or about 25° C., or about 30° C., or about 35° C., or about 40° C., or about 45° C., or about 50° C. In some embodiments, the exposure conditions are about 40° C./75% relative humidity. In some embodiments, the period of time is at least about 1 month, e.g., 1 month, 2 months, 3 months, 4 months, 5 months, 6 months, 7 months, 8 months, 9 months, 10 months, 11 months, 1 year, or more. In some embodiments, the exposure is carried out for a period of time for at least about 6 months, or at least about 8 months, or at least about 10 months, or at least 1 year, or at least 1.5 years, or at least 2 years. The skilled artisan can appreciate that by varying one or more conditions, degradation of a compound, e.g., deuruxolitinib, can be accelerated to form one or more of the disclosed compounds. For example, in some embodiments, increasing the temperature and/or the humidity can accelerate the degradation of deuruxolitinib to form one or more of the disclosed compounds.

In some embodiments, the compounds of Formulas (I)-(VI) are prepared by oxidizing a compound of Formula (VII):

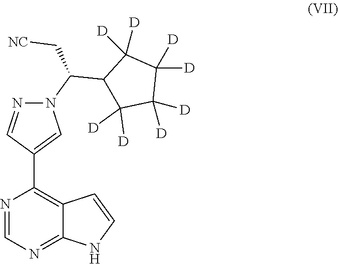 [SMILES: [2H]C1([2H])C([C@@H](CC#N)n2cc(-c3ncnc4[nH]ccc34)cn2)C([2H])([2H])C([2H])([2H])C1([2H])[2H]]

or a pharmaceutically acceptable salt thereof.

In some embodiments, the compounds of Formulas (I)-(VI) are prepared by oxidizing the pharmaceutical salt of the compound of Formula (VIII):

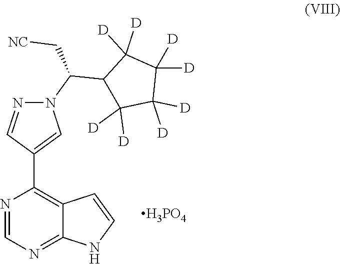

Formula (VII) is also referred to herein as CTP-543, whereas Formula (VIII) is a phosphate salt form (e.g., phosphoric acid salt) of CTP-543. For convenience, as described throughout the present disclosure, the term “CTP-543” can include both the Formula (VII) and Formula (VIII). The method optionally includes the further step of forming a pharmaceutically acceptable salt of the compound of any one of Formulas (I)-(VI). Oxidation conditions can include contact with a peroxide, such as hydrogen peroxide, for a time sufficient to produce a compound of any one of Formulas (I)-(VI) (e.g., from about 2 hours to about 24 hours, such as 6 hours). In some embodiments, the oxidation conditions include contact with a radical initiator, such as azobisisobutyronitrile (“AIBN”) for a time sufficient to produce a compound of any one of Formulas (I)-(VI) (e.g., from about 2 hours to about 24 hours). In some embodiments, the oxidation reaction which produce a compound of any one of Formulas (I)-(VI) can occur over an extended period of time. For example, oxidation conditions can include low concentrations of an oxidizing agent reacting with CTP-543 for an extended period of time, wherein the compound of any one of Formulas (I)-(VI) is produced over the extended period of time, e.g., greater than 1 week, greater than 1 month, greater than 6 months, greater than a year, etc. In some embodiments, the concentration of an oxidizing agent itself can form in a composition over time, and thus form increasing amounts of the compound of any one of Formulas (I)-(VI) over the extended period of time as more oxidizing agent is formed.

### Pharmaceutical Compositions

In some embodiments, provided herein are pharmaceutical compositions comprising a compound of any one of Formulas (I)-(VI), or a pharmaceutically acceptable salt thereof; and a pharmaceutically acceptable carrier. In certain embodiments, the pharmaceutical composition is provided as a unit dose form.

In some embodiments, provided herein is a pharmaceutical composition comprising a pharmaceutically acceptable carrier and a compound represented by the following structural Formula (I):

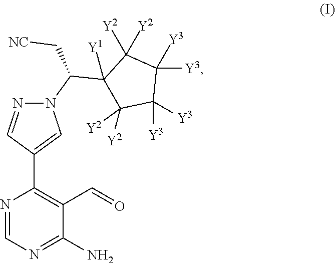

or a pharmaceutically acceptable salt thereof, wherein Y¹ is selected from hydrogen and deuterium; each Y² is independently selected from hydrogen and deuterium; and each Y³ is independently selected from hydrogen and deuterium. In some embodiments, each Y² is the same. In some embodiments, each Y² at one carbon position on the cyclopentyl ring is two hydrogens and each Y² at the other carbon position on the cyclopentyl ring is two deuterium. In some embodiments, at one or both carbon positions on the cyclopentyl ring, one Y² is a hydrogen and one Y² is a deuterium. In some embodiments, each Y³ is the same. In some embodiments, each Y³ at one carbon position on the cyclopentyl ring is two hydrogens and each Y³ at the other carbon position on the cyclopentyl ring is two deuterium. In some embodiments, at one or both carbon positions on the cyclopentyl ring, one Y³ is a hydrogen and one Y³ is a deuterium. In some embodiments, at least one of Y¹, Y², and Y³ is deuterium. In some embodiments, Y¹ is hydrogen. In some embodiments, each Y² and each Y³ is deuterium. In some embodiments, Y¹ is deuterium, and each Y² and each Y³ is deuterium. In some embodiments, Y¹ is hydrogen, each Y² is hydrogen, and each Y³ is deuterium. In some embodiments, Y¹ is hydrogen, each Y² is deuterium, and each Y³ is hydrogen.

In some embodiments, the composition comprises a compound selected from any of Formulas (II)-(VI):

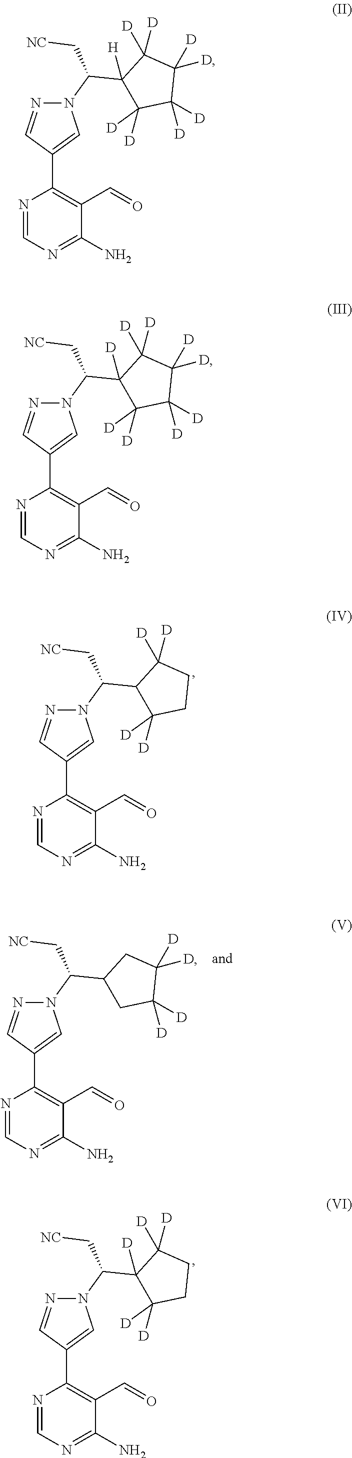

or a pharmaceutically acceptable salt thereof. In some embodiments, the composition comprises a phosphate salt of a compound of any of Formulas (II)-(VI).

In some embodiments, the composition comprises a compound of Formula (II):

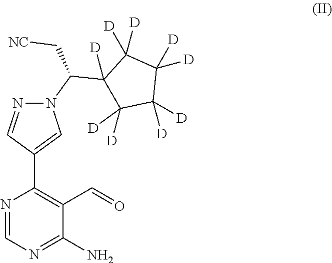 [SMILES: [2H]C1([2H])C([2H])([2H])C([2H])([2H])C([2H])([C@@H](CC#N)n2cc(-c3ncnc(N)c3C=O)cn2)C1([2H])[2H]]

or a pharmaceutically acceptable salt thereof.

The terms pharmaceutically acceptable carriers and vehicles can be used interchangeably and can be used in the pharmaceutical compositions of the disclosure. Pharmaceutically acceptable carriers include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some embodiments, the pharmaceutical composition comprises a pharmaceutically acceptable carrier, wherein the pharmaceutically acceptable carrier is a binder and disintegrant, such as polyvinyl pyrrolidone (povidone). Suitable grades of povidone can include, but are not limited to, povidone K25, povidone K30, povidone K30 LP, povidone K90, povidone C, and the like. In some embodiments, the povidone is K30. In some embodiments, the povidone is povidone K30 LP.

In some embodiments, the pharmaceutical composition comprises povidone. In some embodiments, povidone grades suitable for use herein include, but are not limited to povidone K25 (e.g., Kollidon® 25; BASF Pharma, Germany), povidone K30 (e.g., Kollidon® 30; BASF Pharma, Germany), povidone K30 LP (e.g., Kollidon® 30 LP; BASF Pharma, Germany), povidone K90, povidone C, and the like. In some embodiments, the povidone is povidone K30. In some embodiments, the povidone is a low peroxide povidone, such as povidone K30 LP.

If required, the solubility and bioavailability of the compounds of the present disclosure in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this disclosure optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the disclosure include those suitable for oral administration. Other formulations may conveniently be presented in unit dosage form, e.g., tablets, sustained release capsules, granules, and in liposomes, and may be prepared by any methods well known in the art of pharmacy. See, for example, Remington: The Science and Practice of Pharmacy, Lippincott Williams & Wilkins, Baltimore, MD (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present disclosure suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In some embodiments, the compound is administered orally as a tablet.

In the case of tablets for oral use, carriers that are commonly used include lactose and cornstarch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added. In another embodiment, the composition is in the form of a tablet. In certain embodiments, exemplary formulations for the tablet are disclosed in U.S. Pat. No. 8,754,224, the teachings of which are herein incorporated by reference.

### Combination Pharmaceutical Compositions

In some embodiments, the present disclosure is directed to a pharmaceutical composition comprising (i) a compound of any of Formulas (I)-(VI) or a pharmaceutically acceptable salt thereof; (ii) one or more (e.g., 1, 2 or 3) additional therapeutic agents, e.g., JAK inhibitors, e.g., CTP-543; and (iii) a pharmaceutically acceptable carrier. In some embodiments, the additional therapeutic agent can be a compound of Formula (VII) or Formula (VIII).

In some embodiments, the pharmaceutical composition comprises: (i) povidone K30 LP (ii) a compound of any of Formulas (I)-(VI) or a pharmaceutically acceptable salt thereof at a concentration of about 0.00001% to about 1.0%; and (iii) one or more (e.g., 1, 2 or 3) additional therapeutic agents, e.g., CTP-543 being one of the therapeutic agents. In some embodiments, the compound of any of Formulas (I)-(VI) or a pharmaceutically acceptable salt thereof is present in the pharmaceutical composition at a concentration of about 0.00001% to about 0.5% w/w of the total pharmaceutical composition, or about 0.0001% to about 0.2% w/w of the total pharmaceutical composition, or about 0.001% to about 0.1% w/w of the total pharmaceutical composition. In some embodiments, the compound of any of Formulas (I)-(VI) or a pharmaceutically acceptable salt thereof is present in the pharmaceutical composition at a concentration of about 0.00001% to about 2% w/w of the total pharmaceutical composition, e.g. about 0.001% to about 1%, or about 0.01% to about 0.5% wt/wt of the total pharmaceutical composition. In some embodiments, the additional therapeutic agent is one or more of CTP-543, ruxolitinib, tofacitinib, baricitinib, upadacitinib, fedratinib, filgotinib, momelotinib, pacritinib, itacitinib, peficitinib, PF-06651600, PF-06700841 (brepocitinib), and abrocitinib, or pharmaceutically acceptable salts thereof. In some embodiments, the additional therapeutic agent is CTP-543.

In some embodiments, the ratio of CTP-543 to the compound of any one of Formulas (I)-(V) is about 10:1 to about 10⁸:1, about 100:1 to about 10⁸:1, about 10³:1 to about 10⁸:1, about 10⁴:1 to about 10⁸:1, or about 10⁵:1 to about 10⁸:1. In some embodiments, the amount of the compound of any one of Formulas (I)-(VI) is undetectable using standard techniques, but can be detected when using high precision methods, techniques, and instrumentation, or can be detected when the compound of any one of Formulas (I)-(V) is first concentrated before detection.

In some embodiments, the disclosure provides for pharmaceutical compositions with low amounts of the compounds of Formulas (I)-(VI) when stored for an extended period of time, e.g., for 1 month, 2 months, 3 months, 4 months, 6 months, 9 months, 12 months, 15 months, 18 months, or 2 years. For example, the disclosure provides for a pharmaceutical composition comprising: (i) povidone K30 LP, (ii) a compound of any of Formulas (I)-(VI) or a pharmaceutically acceptable salt thereof at a concentration of about 0.00000001% to about 0.1%; and (iii) CTP-543 when stored for an extended period of time, e.g., for 1 month, 2 months, 3 months, 4 months, 6 months, 9 months, 12 months, 15 months, 18 months, or 2 years at 25° C. and 50% humidity. In some embodiments, the composition comprises (i) povidone K30 LP, (ii) a compound of any of Formulas (I)-(VI) or a pharmaceutically acceptable salt thereof present in the pharmaceutical composition at a concentration of about 0.0000001% to about 0.01% w/w of the total pharmaceutical composition, or about 0.00000001% to about 0.001% w/w of the total pharmaceutical composition, or about 0.00000001% to about 0.001% w/w of the total pharmaceutical composition, and (iii) CTP-543 when stored for an extended period of time, e.g., for 1 month, 2 months, 3 months, 4 months, 6 months, 9 months, 12 months, 15 months, 18 months, or 2 years at 25° C. and 50% humidity. In some embodiments, the disclosure provides a pharmaceutical composition comprising: (i) povidone K30 LP, (ii) the compound of any of Formulas (I)-(VI) or a pharmaceutically acceptable salt thereof is present in the pharmaceutical composition at a concentration of about 0.00000001% to about 0.0001% w/w of the total pharmaceutical composition, e.g. about 0.1%, about 0.05%, about 0.01%, about 0.005%, about 0.001%, about 0.0005% or about 0.0001% to about 1×10⁻⁷%, about 1×10⁻⁶%, or about 1×10⁻⁵%, and (iii) CTP-543 when stored for an extended period of time, e.g., for 1 month, 2 months, 3 months, 4 months, 6 months, 9 months, 12 months, 15 months, 18 months, or 2 years at 25° C. and 50% humidity.

In some embodiments, the disclosure provides for a pharmaceutical composition comprising: (i) povidone K30 LP, (ii) a compound of any of Formulas (I)-(VI) or a pharmaceutically acceptable salt thereof at a concentration of about 0.00000001% to about 0.1%; and (iii) CTP-543 when stored for an extended period of time, e.g., for 1 month, 2 months, 3 months, 4 months, 6 months, 9 months, 12 months, 15 months, 18 months, or 2 years at 40° C. and 75% humidity. In some embodiments, the composition comprises (i) povidone K30 LP, (ii) a compound of any of Formulas (I)-(VI) or a pharmaceutically acceptable salt thereof present in the pharmaceutical composition at a concentration of about 0.0000001% to about 1% w/w of the total pharmaceutical composition, or about 0.00000001% to about 0.5% w/w of the total pharmaceutical composition, or about 0.00000001% to about 0.2% w/w of the total pharmaceutical composition, and (iii) CTP-543 when stored for an extended period of time, e.g., for 1 month, 2 months, 3 months, 4 months, 6 months, 9 months, 12 months, 15 months, 18 months, or 2 years at 40° C. and 75% humidity. In some embodiments, the disclosure provides a pharmaceutical composition comprising: (i) povidone K30 LP, (ii) the compound of any of Formulas (I)-(VI) or a pharmaceutically acceptable salt thereof is present in the pharmaceutical composition at a concentration of about 0.00000001% to about 1% w/w of the total pharmaceutical composition, e.g. about 0.5% to about 0.0001%, about 0.2% to about 0.001%, or about-0.1% to about 0.01%, and (iii) CTP-543 when stored for an extended period of time, e.g., for 1 month, 2 months, 3 months, 4 months, 6 months, 9 months, 12 months, 15 months, 18 months, or 2 years at 40° C. and 75% humidity.

In some embodiments, the pharmaceutical composition is formulated as a tablet or capsule. In some embodiments, the pharmaceutical composition is formulated as a tablet.

In some embodiments, the pharmaceutical composition comprises (i) povidone K30 LP; (ii) less than 0.05% w/w Formula (I) or a pharmaceutically acceptable salt thereof; (iii) and CTP-543. In some embodiments, the pharmaceutical composition comprises (i) povidone K30 LP; (ii) less than 0.05% w/w Formula (II) or a pharmaceutically acceptable salt thereof; (iii) and CTP-543. In some embodiments, the pharmaceutical composition comprises (i) povidone K30 LP; (ii) less than 0.05% w/w Formula (III) or a pharmaceutically acceptable salt thereof; (iii) and CTP-543. In some embodiments, the pharmaceutical composition comprises (i) povidone K30 LP; (ii) less than 0.05% w/w Formula (IV) or a pharmaceutically acceptable salt thereof; (iii) and CTP-543. In some embodiments, the pharmaceutical composition comprises (i) povidone K30 LP; (ii) less than 0.05% w/w Formula (V) or a pharmaceutically acceptable salt thereof; (iii) and CTP-543. In some embodiments, the pharmaceutical composition comprises (i) povidone K30 LP; (ii) less than 0.05% w/w Formula (VI) or a pharmaceutically acceptable salt thereof; (iii) and CTP-543. In some embodiments, the pharmaceutical composition comprises (i) povidone K30 LP; (ii) less than 0.005% w/w Formula (I) or a pharmaceutically acceptable salt thereof; (iii) and CTP-543. In some embodiments, the pharmaceutical composition comprises (i) povidone K30 LP; (ii) less than 0.005% w/w Formula (II) or a pharmaceutically acceptable salt thereof; (iii) and CTP-543. In some embodiments, the pharmaceutical composition comprises (i) povidone K30 LP; (ii) less than 0.005% w/w Formula (III) or a pharmaceutically acceptable salt thereof; (iii) and CTP-543. In some embodiments, the pharmaceutical composition comprises (i) povidone K30 LP; (ii) less than 0.005% w/w Formula (IV) or a pharmaceutically acceptable salt thereof; (iii) and CTP-543. In some embodiments, the pharmaceutical composition comprises (i) povidone K30 LP; (ii) less than 0.005% w/w Formula (V) or a pharmaceutically acceptable salt thereof; (iii) and CTP-543. In some embodiments, the pharmaceutical composition comprises (i) povidone K30 LP; (ii) less than 0.005% w/w Formula (VI) or a pharmaceutically acceptable salt thereof; (iii) and CTP-543.

In some embodiments, the pharmaceutical composition comprises (i) a compound of Formulas (I)-(VI), or a pharmaceutically acceptable salt thereof; (ii) CTP-543; and (iii) povidone K30 LP, wherein the amount of a compound of Formulas (I)-(VI) is greater than 0.000001% of the composition, greater than 0.00001% of the composition, greater than 0.0001% of the composition, greater than 0.001% of the composition, greater than 0.01% of the composition, greater than 0.05% of the composition or greater than 0.1% of the composition. In some embodiments, the amount of a compound of Formulas (I)-(VI) is determined 1 week, 1 month, 3 months, 6 months, 9 month, 1 year, 18 months, or 2 years after formation of the composition. Thus, the disclosure provides that a composition may have an undetectable concentration of the compounds of Formulas (I)-(VI) at an initial time point, but then may form a compound of Formulas (I)-(VI) at a later time point.

In some embodiments, the disclosure provides a pharmaceutical formation comprising CTP-543, wherein the CTP-543 is stable, e.g., less than 1%, less than 0.5%, less than 0.1%, less than 0.05%, or less than 0.01% of the CTP-543 is oxidized to form a compound of Formulas (I)-(VI). In some embodiments, the disclosure provides a pharmaceutical formulation comprising CTP-543 and a pharmaceutical carrier, wherein the CTP-543 is stable, e.g., less than 1%, less than 0.5%, less than 0.1%, less than 0.05%, or less than 0.01% of the CTP-543 is oxidized to form a compound of Formulas (I)-(VI) at 1 week, 1 month, 3 months, 6 months, 9 months, 1 year, 18 months or 2 years after the pharmaceutical formulation is formed, e.g., at 25° C. and 50% humidity, and/or at 40° C. and 75% humidity.

In some embodiments, the pharmaceutical composition is in a unit dosage form, e.g., a capsule or table, wherein the unit dosage form comprises an amount of CTP-543 of 4 mg, 8 mg, 12 mg, 16 mg, or 24 mg. In some embodiments, the amount of one or more compounds of Formula (I)-(VI) and the amount of CTP-543 together in the pharmaceutical composition is 4 mg, 8 mg, 12 mg, 16 mg, or 24 mg. In some embodiments, the pharmaceutically acceptable carrier is a binder, such as povidone. In some embodiments, the povidone is povidone K30 LP. In some embodiments, the Formula (I), (II), (III, (IV), (V), or (VI) is present in the pharmaceutical composition at a concentration of less than 0.05%, or less than 0.01%, or less than 0.005%, or less than 0.00005% w/w.

The interrelationship of dosages for animals and humans (based on milligrams per meter squared of body surface) is described in Freireich et al., Cancer Chemother. Rep, 1966, 50:219. Body surface area may be approximately determined from height and weight of the subject. See, e.g., Scientific Tables, Geigy Pharmaceuticals, Ardsley, N.Y., 1970, 537.

It will be understood that reference to an amount of a compound of any of Formulas (I)-(VI), or a pharmaceutically acceptable salt thereof, includes an amount of a pharmaceutically acceptable salt of the compound of Formula (I)-(VI) (such as the phosphate salt) which is equivalent to the stated amount of the compound of Formula (I)-(VI) as the free base. For example, about 20.7 mg of Compound (II) phosphate salt is equivalent to 16 mg of Compound (II) free base.

In some embodiments, the pharmaceutical composition comprises an effective amount of a composition comprising (i) a compound of Formula (I), or a pharmaceutically acceptable salt thereof; (ii) an additional therapeutic agent; and a pharmaceutically acceptable carrier. In some embodiments, the pharmaceutical composition comprises: (i) a compound of Formula (I) or a pharmaceutically acceptable salt thereof; (ii) one or more JAK inhibitors; and a pharmaceutically acceptable carrier. Examples of JAK inhibitors include, but are not limited to, CTP-543, ruxolitinib, tofacitinib, baricitinib, upadacitinib, fedratinib, filgotinib, momelotinib, pacritinib, itacitinib, peficitinib, PF-06651600, PF-06700841 (brepocitinib), and abrocitinib, and pharmaceutically acceptable salts thereof. In some embodiments, the JAK inhibitor is CTP-543, or a pharmaceutically acceptable salt thereof. In some embodiments, the composition comprises about 8 mg, 12 mg, 16 mg, 24 mg, or 32 mg of CTP-543.

In another embodiment, provided herein is a unit dose form comprising a compound of Formula (1), or a pharmaceutically acceptable salt thereof; and a pharmaceutically acceptable carrier. In some embodiments, the unit dose form comprises a compound of Formula (I), or a pharmaceutically acceptable salt thereof, together with CTP-543 or a pharmaceutically acceptable salt thereof. In some embodiments, the amount of CTP-543, or a pharmaceutically acceptable salt thereof, in the unit dose form is about 8 mg, 12 mg, 16 mg, or 24 mg. In some embodiments, the amount of CTP-543, or a pharmaceutically acceptable salt thereof, in the unit dose form is about 8 mg or about 12 mg. In some embodiments, the amount of CTP-543, or a pharmaceutically acceptable salt thereof, in the unit dose form is about 16 mg or about 24 mg. In some embodiments, the amount of the compound of Formula (I), or a pharmaceutically acceptable salt thereof, is between 1 mg and 50 mg. In some embodiments, the unit dose form is a tablet. In some embodiments, the unit dose form is a capsule.

### Methods of Treatment

Also provided herein is a method of modulating the activity of JAK1 and/or JAK2 in a cell, comprising contacting a cell with a composition comprising a JAK inhibitor, e.g., CTP-543, wherein the composition further comprises a compound of Formulas (I)-(VI) as described herein, or a pharmaceutically acceptable salt thereof. In some embodiments, the method of modulating the activity of JAK and/or JAK2 in a cell comprising contacting a cell with a composition comprising a JAK Inhibitor, e.g., CTP-543, wherein the composition further comprises a compound of Formula (II) herein, or a pharmaceutically acceptable salt thereof. In some embodiments, the method of modulating the activity of JAK and/or JAK2 in a cell comprising contacting a cell with a composition comprising a JAK Inhibitor, e.g., CTP-543, wherein the composition further comprises a compound of Formula (III) herein, or a pharmaceutically acceptable salt thereof. In some embodiments, the method of modulating the activity of JAK and/or JAK2 in a cell comprising contacting a cell with a composition comprising a JAK Inhibitor, e.g., CTP-543, wherein the composition further comprises a compound of Formula (IV) herein, or a pharmaceutically acceptable salt thereof. In some embodiments, the method of modulating the activity of JAK and/or JAK2 in a cell comprising contacting a cell with a composition comprising a JAK Inhibitor, e.g., CTP-543, wherein the composition further comprises a compound of Formula (V) herein, or a pharmaceutically acceptable salt thereof. In some embodiments, the method of modulating the activity of JAK and/or JAK2 in a cell comprising contacting a cell with a composition comprising a JAK Inhibitor, e.g., CTP-543, wherein the composition further comprises a compound of Formula (VI) herein, or a pharmaceutically acceptable salt thereof.

In some embodiments, the cell is contacted in vitro. In some embodiments, the cell is contacted in vivo. In some embodiments, the cell is contacted ex vivo.

In some embodiments, provided herein is a method of treating a disease or disorder that is beneficially treated by compounds that modulate (e.g., inhibit) the activity of a JAK (JAK1, JAK2, JAK3 and/or TYK2) in a subject in need thereof, comprising the step of administering to the subject a pharmaceutical composition comprising a composition comprising a JAK Inhibitor, e.g., CTP-543, wherein the composition further comprises a compound of Formula (I) or a pharmaceutically acceptable salt thereof. In some embodiments the subject is a patient in need of such treatment. Such diseases, conditions or disorders include, without limitation, skin diseases such as proliferative, autoimmune and/or inflammatory skin disorders including psoriasis, atopic dermatitis, scleroderma, rosacea, skin cancers, dermatitis, dermatitis herpetiformis, dermatomyositis, vitiligo, hair loss disorders, contact dermatitis, xerosis, ichthyosis, hidradenitis suppurativa, urticaria, lichen planus, prurigo nodularis, vasculitis, cutaneous lupus erythematosus (CLE), and chronic idiopathic pruritus; hyperproliferative disorders or cancers including polycythemia vera, essential thrombocytopenia, and myelofibrosis; respiratory diseases such as asthma, chronic obstructive pulmonary disease, chronic lung allograft dysfunction, e.g., bronchiolitis obliterans syndrome, pulmonary fibrosis, cystic fibrosis, rhinitis, bronchiolitis, byssinosis, pneumoconiosis, bronchiectasis, hypersensitivity pneumonitis, lung cancers, mesothelioma and sarcoidosis; gastrointestinal diseases such as inflammatory bowel disease, ulcerative colitis, Crohn's disease, retroperitoneal fibrosis, celiac disease and cancers; eye diseases such as myasthenia gravis, Sjogren's syndrome, conjunctivitis, scleritis, uveitis, Giant cell arteritis, Discoid lupus, pyoderma gangrenosum, Bollus skin disease, Idiopathic pulmonary disease, dry eye syndrome, keratitis, iritis; systemic indications such as lupus, multiple sclerosis, rheumatoid arthritis, type I diabetes and complications from diabetes, cancers, ankylosing spondylitis and psoriatic arthritis; as well as other autoimmune diseases and indications where immunosuppression would be desirable, for example, to treat or prevent acute and/or chronic graft-versus-host disease (e.g., in organ transplantation). In some embodiments, a disease or condition mediated alone, or in part, by JAK signaling is a hair loss disorder, such as alopecia areata.

In some embodiments, provided herein is a method for treating hair loss disorders that can be treated by compounds that modulate (e.g., inhibit) the activity of a JAK (JAK1, JAK2, JAK3 and/or TYK2). In some embodiments, the method comprises administering to a mammalian (e.g., human) subject a pharmaceutical composition comprising a composition comprising a JAK Inhibitor, e.g., CTP-543, wherein the composition further comprises a compound of Formulas (I)-(VI), or a pharmaceutically acceptable salt thereof. In some embodiments, the method comprises administering to a mammalian (e.g., human) subject a pharmaceutical composition comprising a composition comprising a JAK Inhibitor, e.g., CTP-543, wherein the composition further comprises a compound of Formula (II), or Formula (III), or Formula (IV), or Formula (V), or Formula (VI), or a pharmaceutically acceptable salt thereof.

In some embodiments, the method for treating hair loss disorders comprises administering a therapeutically effective amount of a composition comprising a JAK Inhibitor, e.g., CTP-543, wherein the composition further comprises a compound of Formula (I)-(VI). In some embodiments, the CTP-543 is administered to a subject in an amount of 4 mg/day, 8 mg/day, 12 mg/day, 16 mg/day, 24 mg/day, 32 mg/day, or 48 mg/day. In some embodiments, the method comprises administering the composition comprising a JAK Inhibitor, e.g., CTP-543, wherein the composition further comprises a compound of Formula (I)-(VI) at 4 mg twice a day, 8 mg once a day, 8 mg twice a day, 12 mg once a day, 12 mg twice a day, 16 mg once a day, 16 mg twice a day, 24 mg once a day, or 24 mg twice a day.

Hair loss disorders as provided herein can include, without limitation, androgenetic alopecia, alopecia areata, ophiasis, telogen effluvium, alopecia totalis, and alopecia universalis. The term alopecia areata can include alopecia totalis and alopecia universalis.

Alopecia areata is an autoimmune disease that can result in partial or complete loss of hair on the scalp and body. In some embodiments, the disclosure provides for compositions suitable for the treatment of a hair loss disorder of the scalp. The scalp is the most commonly affected area, but any hair-bearing site can be affected alone or together with the scalp and can be treating using the compositions described herein. Onset of the disease can occur throughout life and affects both women and men. Alopecia areata can be associated with serious psychological consequences, including anxiety and depression. Thus, in some embodiments, the compositions described herein can be used in methods of treating serious psychological conditions, including anxiety and depression

In some embodiments, the condition is alopecia areata in a subject such as a mammalian (e.g., human) patient in need thereof. In some embodiments, the alopecia areata is moderate to severe alopecia areata (for example, hair loss over at least 30% of the scalp, hair loss over at least 40% of the scalp, or hair loss over at least 50% of the scalp).

In some embodiments, the compound or compositions of the disclosure can be used to treat loss of facial hair, including eyebrows, eyelashes, nose hair, mustache, and/or beard.

In some embodiments, the compound or compositions of the disclosure can be used to treat other manifestations of alopecia areata, including scalp pain or sensitivity, or malformed fingernails or toenails.

In some embodiments, a compound or compositions of the disclosure can be of the disclosure is administered orally once a day. In some embodiments, a compound or compositions of the disclosure can be of the disclosure is administered orally twice per day. In some embodiments, a pharmaceutical composition of the disclosure (e.g., comprising a compound any one of Formulas (I)-(VI) and a JAK inhibitor, e.g., CTP-543) is administered orally once a day. In some embodiments, a pharmaceutical composition of the disclosure (e.g., comprising a compound any one of Formulas (I)-(VI) and a JAK inhibitor, e.g., CTP-543) is administered orally twice per day.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician.

The administration of a composition comprising CTP-543 and a compound of Formula (I), or a pharmaceutically acceptable salt thereof (such as the phosphate salt), can continue for as long as necessary to treat a hair loss disorder, e.g., for one week, two weeks, one month, two months, three months, four months, six months, one year, two years, five years, ten years, or longer.

In some embodiments, the compositions comprising the compounds described herein can be used in treating autoimmune, inflammatory, and cancer diseases mediated by dysregulation of JAK activity.

The efficacy of treatment of hair loss disorders such as alopecia areata can be measured in a variety of ways, some of which are known in the art. For example, the “severity of alopecia tool,” otherwise known as SALT, is a validated assessment scale (developed by the National Alopecia Areata Foundation working committee) to evaluate the degree of hair loss. See, e.g., Olsen E A, Hordinsky M K, Price V H, et al. Alopecia areata investigational assessment guidelines-Part II. J Am Acad Dermatol 2004:51: 440-447 (incorporated herein by reference). The SALT score is calculated for a subject (e.g., human patient) by measuring the percentage of hair loss in each of the 4 areas of the scalp and adding the total to achieve a composite score. Hair regrowth is reflected by a decrease in the SALT score. For example, no hair on the scalp would have a SALT score of 100 while complete hair regrowth would be a SALT score of 0. In some embodiments, methods of treatment as described herein can provide a SALT score improvement of at least 10 points after treatment (for example, from a SALT score of 100 prior to treatment to a SALT score of 90 after treatment). In some embodiments, methods of treatment as described herein can provide a SALT score improvement of at least 20 points, 30 points, 40 points, 50 points, 60 points, 70 points, 80 points, 90 points, or 100 points. In some embodiments, methods of treatment as described herein can provide after treatment at least a 20% improvement from baseline in the subject's SALT score, or at least a 30% improvement from baseline in the subject's SALT score, or at least a 40% improvement from baseline in the subject's SALT score, or at least a 50% improvement from baseline in the subject's SALT score, or at least a 60% improvement from baseline in the subject's SALT score, or at least a 70% improvement from baseline in the subject's SALT score.

In some embodiments, treatment is continued for a period of at least four weeks, or at least 8 weeks, or at least 12 weeks, or at least 16 weeks, or at least 20 weeks, or at least 24 weeks, or at least 28 weeks, or at least 32 weeks, or at least 36 weeks, or at least 40 weeks, or at least 44 weeks, or at least 48 weeks, or at least 52 weeks. In some embodiments, the methods of treatment described herein can provide a SALT score improvement of at least 20 points after the treatment is continued for a period of at least 8 weeks, or at least 12 weeks, or at least 16 weeks, 24 weeks, or at least 28 weeks, or at least 32 weeks, or at least 36 weeks, or at least 52 weeks. In some embodiments, the SALT score improvement of at least 20 points is provided after the treatment is continued for a period of at least 24 weeks. In some embodiments, the methods of treatment described herein can provide a SALT score improvement of at least 50 points after the treatment is continued for a period of at least 24 weeks, or at least 28 weeks, or at least 32 weeks, or at least 36 weeks, or at least 52 weeks. In some embodiments, the SALT score improvement of at least 50 points is provided after the treatment is continued for a period of at least 24 weeks. In some embodiments, the methods of treatment described herein can provide a SALT score improvement of at least 80 points after the treatment is continued for a period of at least 52 weeks. In some embodiments, the methods of treatment described herein can provide at least a 20% improvement from baseline in the subject's SALT score after the treatment is continued for a period of at least 8 weeks, or at least 16 weeks, or at least 24 weeks. In some embodiments, the methods of treatment described herein can provide at least a 20% improvement from baseline in the subject's SALT score after the treatment is continued for a period of at least 8 weeks.

In some embodiments, the pharmaceutical composition is in the form of a tablet. In an exemplary embodiment, the table comprises the following ingredients after 10 months in storage at 40° C./75% RH):

TABLE 1A
8 mg strength CTP-543 tablet (contains equivalent of
8 mg free base deuruxolitinib).

| Component | Wt % |
|---|---|
| CTP-543 (phosphate) | 8.75 |
| Compound of Formula (II) | 0.07% to 0.08% |
| Microcrystalline cellulose | 33.25 |
| (intragranular) | |
| Microcrystalline cellulose | 19 |
| (extragranular) | |
| Lactose monohydrate | 30 |
| Povidone K30 | 5 |
| Hydroxypropyl cellulose | 3 |
| Colloidal silicon dioxide | 0.5 |
| Magnesium stearate | 0.5 |
| Total (tablet core) | 100 |
| Opadry ® amb II | |
| Total (coated tablet) | |

Thus, in some embodiments, the disclosure provides a tablet comprising a pharmaceutical composition, wherein the pharmaceutical composition comprises the following after 10 months in storage at 40° C./75% RH) (Table 1B):

TABLE 1B

| Component | Wt % |
|---|---|
| CTP-543 (phosphate) | 8.75 |
| Formula (II) compound | 0.02% to 0.1% |
| Povidone K30 | 1%-20% |
| Other pharmaceutical carriers | 91% to 72% |
| Total (tablet core) | 100 |
| Opadry ® amb II | |
| Total (coated tablet) | |

In some embodiments, the disclosure provides a tablet comprising a pharmaceutical composition, wherein the pharmaceutical composition comprises the following after 10 months in storage at 40° C./75% RH) (Table 1C):

TABLE 1C

| Component | Wt % |
|---|---|
| CTP-543 (phosphate) | 8.75 |
| Formula (II) compound | Less than 0.01% |
| Povidone K30 LP | 1%-20% |
| Other pharmaceutical carriers | 91% to 72% |
| Total (tablet core) | 100 |
| Opadry ® amb II | |
| Total (coated tablet) | |

In some embodiments, an additional active agent can be administered with the compounds or compositions described herein. The choice of additional therapeutic agent is dependent upon the particular disease or condition to be treated. For the treatment of hair loss, the choice of additional therapeutic agent may be made from any additional therapeutic agent known to be useful for treatment of hair loss disorders such as alopecia areata. Examples of additional therapeutic agents that may be employed in the methods of this disclosure are those set forth above for use in combination compositions comprising a compound of Formula (I), or a pharmaceutically acceptable salt thereof, and an additional therapeutic agent. Additional therapeutic agents include agents used in the treatment of alopecia areata, including, for example, topical minoxidil, injected corticosteroids, and anthralin cream or ointment.

The term “co-administered” as used herein means that the additional therapeutic agent may be administered together with a compound of Formula (I) or a pharmaceutically acceptable salt thereof, as part of a single dosage form (such as a pharmaceutical composition of this disclosure comprising the compound of Formula (I), or a pharmaceutically acceptable salt thereof, and an additional therapeutic agent as described above) or as separate, multiple dosage forms. Alternatively, the additional agent may be administered prior to, consecutively with, or following the administration of a composition comprising a compound of Formula (I), or a pharmaceutically acceptable salt thereof. In such combination therapy treatment, both the composition comprising a compound of Formula (1), or a pharmaceutically acceptable salt thereof, and the additional therapeutic agent(s) are administered by conventional methods. The administration of a pharmaceutical composition of this disclosure, comprising both a compound of Formula (I), or a pharmaceutically acceptable salt thereof, and an additional therapeutic agent, to a subject does not preclude the separate administration of that same therapeutic agent, any other additional therapeutic agent or a compound of Formula (I), or a pharmaceutically acceptable salt thereof, to said subject at another time during a course of treatment.

In yet another aspect, the disclosure provides the use of a composition comprising a compound of Formula (I), or a pharmaceutically acceptable salt thereof, alone or together with one or more of the above-described additional therapeutic agents in the manufacture of a medicament, either as a single composition or as separate dosage forms, for treatment or prevention in a subject of a disease, disorder or symptom set forth above. Also provided herein is a composition comprising a compound of Formula (I), or a pharmaceutically acceptable salt thereof, for use in the treatment or prevention in a subject of a disease, disorder or symptom thereof delineated herein.

Various modifications of the disclosure in addition to those shown and described herein will become apparent to those skilled in the art from the foregoing description and fall within the scope of the appended claims. Preferred features of each aspect of the disclosure are as for each of the other aspects mutatis mutandis. The documents referred to herein are incorporated by reference to the fullest extent permitted by law.

### EXAMPLES

### Example 1. Synthesis of the Compound of Formula (II)

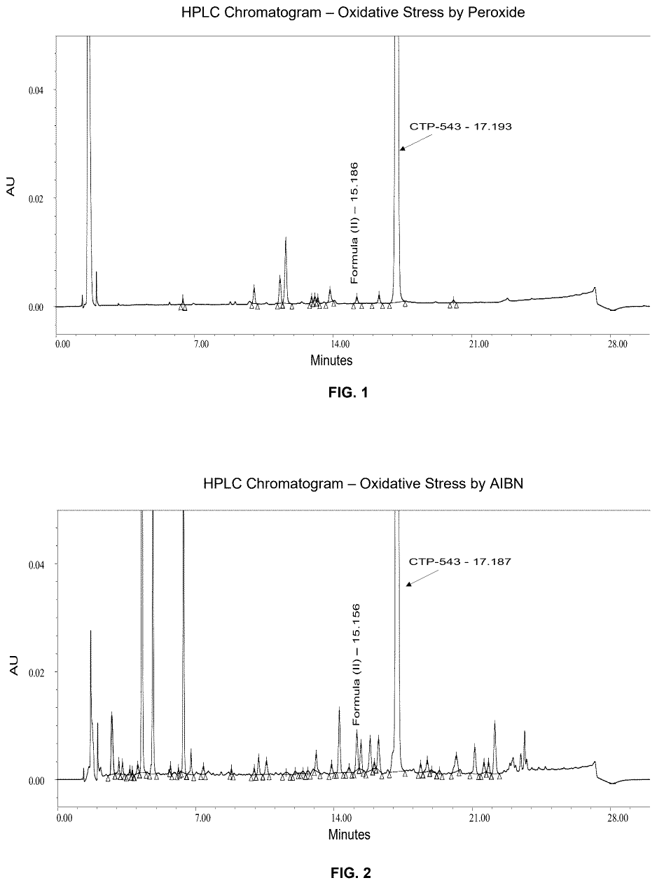

The synthesis of a compound of Formula (II), or a pharmaceutically acceptable salt thereof (such as the phosphate salt) may be readily achieved, e.g., reaction of CTP-543 under conditions suitable to provide hydrolysis and oxidation of the pyrrole ring of CTP-543. CTP-543 can be prepared, e.g., according to the methods described in U.S. Pat. No. 9,249,149 and US Patent Pub. No. 2019/0160068 (the teachings of which are incorporated herein by reference), to produce CTP-543 and/or its phosphate salt. In a first instance, the compound of Formula (II) or its phosphate salt was formed from CTP-543 via an oxidation reaction, e.g., oxidation by a peroxide, such as hydrogen peroxide. Briefly, CTP-543 was mixed with a solution containing 10% v/v hydrogen peroxide at 40° C. for a period of 6 hours to produce the compound of Formula (II). The Formula (II) product was filtered and dried to yield the desired compound. The product was analyzed by reverse-phase HPLC with UV detection (see FIG. 1) and NMR (data not shown).

HPLC method summary: the column was a Waters XBridge C18, 3.5 μm, 4.6×150 mm, gradient elution: mobile phase A was 20 mM ammonium acetate in water, pH 8.55; mobile phase B was acetonitrile; mobile phase C was isopropanol; column temperature was 30° C.; detection was by ultraviolet absorbance at wavelength 254 nm; flow rate was 1.0 mL/minute with a run time of 30 minutes. Result: Formula (II) retention time=15.186 min.

In a second instance, the compound of Formula (II) or its phosphate salt was formed from CTP-543 by reacting CTP-543 with azobisisobutyronitrile (“AIBN”). Briefly, CTP-543 was mixed with a solution containing 5% w/w AIBN at 60° C. for a period of 24 hours. The product was filtered and dried to yield the desired compound. The formula (II) product was analyzed by reverse-phase HPLC with UV detection (see FIG. 2). Result: Formula (II) retention time=15.156 min.

### Example 2. Accumulation of the Compound of Formula (II)

Open-dish studies of 8 mg and 12 mg CTP-543 tablets were carried out in unsealed bottles at 40° C. and 75% relative humidity over a period of 10 months. The Formula (II) compound was detected and eluted at a relative retention time of 0.89 using HPLC. The results are summarized in Table 2.

TABLE 2
Results from open-dish study.

| | 8 mg tablet | 12 mg tablet. |
|---|---|---|
| Formula (II) | 0.08% w/w | 0.07% w/w |
| compound | | |

1. A compound represented by Formula (I) 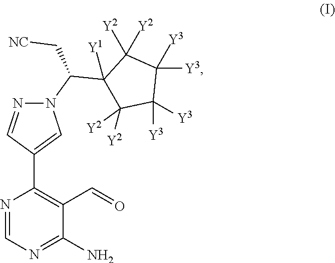
or a pharmaceutically acceptable salt thereof, wherein
Y¹ is selected from hydrogen and deuterium;
each Y² is independently selected from hydrogen and deuterium; and
each Y³ is independently selected from hydrogen and deuterium;
provided that at least one of Y¹, Y², and Y³ is deuterium; and
wherein each position designated specifically as deuterium has at least 95% deuterium incorporation.

2. The compound of claim 1, wherein each Y² is the same.

3. The compound of claim 1, wherein each Y³ is the same.

4. The compound of claim 1, which is a compound selected from any of Formulas (II)-(VI): 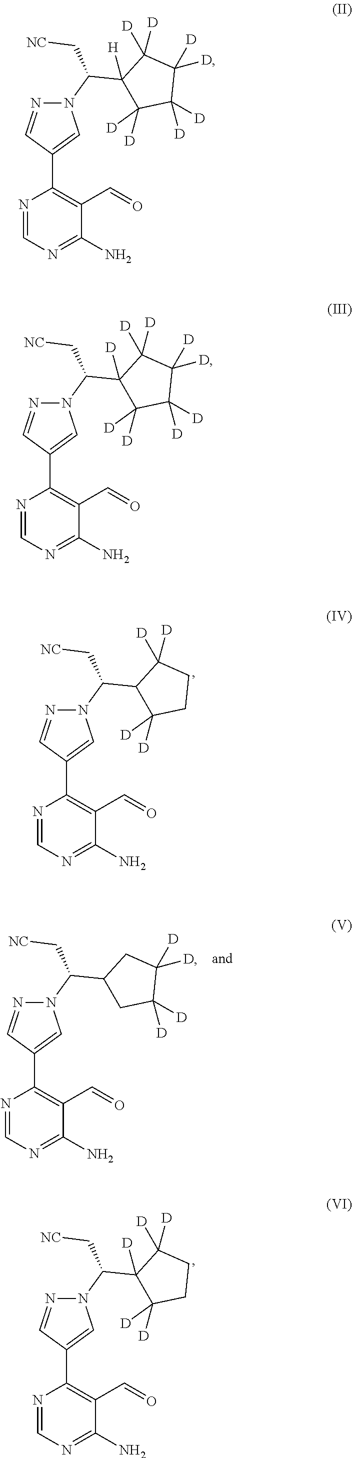
or a pharmaceutically acceptable salt thereof.

5. The compound of claim 4, which is a compound of Formula (II): 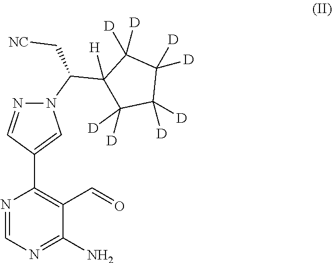 [SMILES: [2H]C1([2H])C([C@@H](CC#N)n2cc(-c3ncnc(N)c3C=O)cn2)C([2H])([2H])C([2H])([2H])C1([2H])[2H]]
or a pharmaceutically acceptable salt thereof.

6. The compound of claim 1, wherein each position designated specifically as deuterium in Formula (I) has at least 97% deuterium incorporation.

7. The compound of claim 1, wherein the pharmaceutically acceptable salt is a phosphate salt.

8. A pharmaceutical composition comprising a compound represented by the following structural formula: 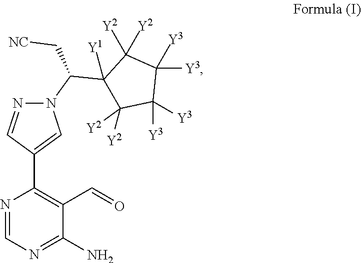
or a pharmaceutically acceptable salt thereof,
wherein Y¹ is selected from hydrogen and deuterium;
each Y² is independently selected from hydrogen and deuterium, wherein each Y² is the same;
each Y³ is independently selected from hydrogen and deuterium, wherein each Y³ is the same;
provided that at least one of Y² and Y³ is deuterium; and
wherein each position designated specifically as deuterium has at least 95% deuterium incorporation; and a pharmaceutically acceptable carrier.

9. (canceled)

10. (canceled)

11. The pharmaceutical composition of claim 8, further comprising an additional therapeutic agent which is a JAK inhibitor.

12. The pharmaceutical composition of claim 11, wherein the additional therapeutic agent which is a JAK inhibitor is selected from CTP-543, ruxolitinib, tofacitinib, baricitinib, upadacitinib, fedratinib, filgotinib, momelotinib, pacritinib, itacitinib, peficitinib, PF-06651600, PF-06700841 (brepocitinib), and abrocitinib, or a pharmaceutically acceptable salt thereof.

13. The pharmaceutical composition of claim 8, wherein the composition comprises a compound of Formula (II) 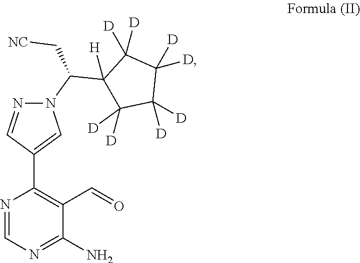
or a pharmaceutically acceptable salt thereof, and CTP-543, or a pharmaceutically acceptable salt thereof.

14. (canceled)

15. The pharmaceutical composition of any one of claim 8, wherein the pharmaceutical composition is formulated in tablet form.

16. The pharmaceutical composition of claim 8, wherein each position designated specifically as deuterium in Formula (I) has at least 97% deuterium incorporation.

17. The pharmaceutical composition of claim 8, wherein the compound of Formula (I) is present in an amount less than 1% w/w of the pharmaceutical composition.

18. (canceled)

19. The pharmaceutical composition of claim 13, further comprising povidone.

20. The pharmaceutical composition of claim 19, wherein the povidone is povidone K30 LP.

21. The pharmaceutical composition of claim 20, wherein the compound of Formula (II) is present in an amount less than 0.05% w/w of the pharmaceutical composition.

22. A method of inhibiting the activity of one or more of JAK1 or JAK2 in a cell, comprising contacting the cell with a pharmaceutical composition of claim 13.

23. (canceled)

24. A method of treating a disease or disorder in a human subject that is beneficially treated by inhibiting the activity of a JAK, the method comprising administering to the subject an effective amount of pharmaceutical composition of claim 13.

25-27. (canceled)

28. A method of treating a disease or disorder that is beneficially treated by inhibiting the activity of a JAK in a human subject in need thereof, comprising administering to the human subject an effective amount of a pharmaceutical composition pharmaceutical composition of claim 13.

29-34. (canceled)
